(19) United States
(12) Patent Application Publication
(10) Pub. No.: US 2009/0137645 A1
(43) Pub. Date: May 28, 2009

(54) Shelf-Stable Famotidine Granulates for Oral Suspensions

(75) Inventors: Feng Zhang, Austin, TX (US); Bryce M. Harvey, Pike Road, AL (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: MIDLOTHIAN LABORATORIES, LLC, Montgomery, AL (US)

(21) Appl. No.: 12/260,552

(22) Filed: Oct. 29, 2008

Related U.S. Application Data
(60) Provisional application No. 60/984,034, filed on Oct. 31, 2007.

(51) Int. Cl. A61K 31/426

(52) U.S. Cl. 514/370

(57) ABSTRACT

A granulate for oral suspension that is particularly useful for the storage and reconstitution of famotidine into a liquid suspension.

### RELATION TO PRIOR APPLICATIONS

The present application claims priority to U.S. provisional application No. 60/984,034, filed Oct. 31, 2007. The application is incorporated herein by reference.

### FIELD OF THE INVENTION

This invention relates to stable granulates of famotidine for oral suspension, the use of the granulates in the production of famotidine suspensions, and the manufacture of such granulates.

### BACKGROUND OF THE INVENTION

Famotidine is chemically known as N′-(aminosulfonyl)-3-[[[2-[(diaminomethylene)amino]-4-thiazolyl]methyl]thio]propanimidamide, and has the following chemical structure:

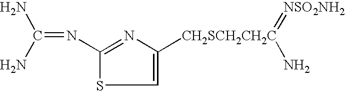

The compound exists as two polymorphic forms - Form A and Form B, having a melting temperature of 172° C. and 163° C. respectively when measured by differential scanning calorimetry. Form B is the preferred form for commercial formulations.

Famotidine is a competitive histamine H₂-receptor antagonist used to treat gastrointestinal disorders such as gastric or duodenal ulcer, gastroesophageal reflux disease, and pathological hypersecretory conditions. Famotidine inhibits many of the isoenzymes of the hepatic CYP450 enzyme system. Famotidine is marketed commercially in the United States as Pepcid®, as immediate release oral tablets, rapidly disintegrating tablets, powders for reconstitution as an oral suspension, and injectable solutions.

Several formulations famotidine are reported in the patent literature, including powders for oral suspension (i.e. formulations which are sold as powders and reconstituted at the pharmacy shortly before use). For example, U.S. Pat. No. 5,593,696 (McNally et al.) describes a stabilized granulate composition of famotidine and sucralfate in which the famotidine is present in a coated granule to avoid contact with the sucralfate. U.S. Pat. No. 5,272,137 (Blase et al.) report a liquid suspension of acetaminophen, also useful for famotidine, made in a series of steps using defined ratios of xanthan gum as the suspending agent and microcrystalline cellulose.

There is a need for robust commercial formulations of famotidine powders and granulates, which can be reconstituted at the pharmacy into a suspension as needed.

### SUMMARY OF THE INVENTION

After intensive study and research, the inventors have developed a dry granular formulation of famotidine that is stable over prolonged periods of time, and that can be reconstituted as a suspension when mixed with water. The formulation employs an organic or mineral acid, preferably citric acid, to impart optimum stability when the product is formed into a suspension, but separates the famotidine from citric acid and water during storage, to avoid unwanted degradation that occurs when the famotidine and citric acid are combined in a wet granulation process.

Accordingly, in a first principal embodiment the invention provides a granulate for oral suspension comprising a first granulate and a second granulate, (a) said first granulate comprising famotidine in the absence of an organic or mineral acid; (b) said second granulate comprising one or more organic or mineral acids in the absence of famotidine; and (c) said first granulate and said second granulate being present at a ratio effective to produce a pH in water of from 6.5 to 7.5 when said granulate for oral suspension is mixed with water at a ratio of about 40 mg. of famotidine per 5 ml of water. In one embodiment, the second granulate further includes preservatives for the composition, though in other embodiments the preservatives can be incorporated into a third or subsequent granulate.

In a second principal embodiment, the invention employs a dry granulation process so that famotidine is not exposed to water during the manufacturing process. In this embodiment, the invention provides a granulate for oral suspension comprising a first granulate that comprises famotidine in combination with an organic or mineral acid, wherein said first granulate has the morphology of a dry granulate, said famotidine and organic or mineral acid being present at a ratio effective to produce a pH in water of from 6.5 to 7.5 when said granulate for oral suspension is mixed with water at a ratio of about 40 mg. of famotidine per 5 ml of water.

The invention further provides methods of using the formulations to reconstitute suspensions, and methods of making the formulations. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

FIG. 1 contains a flow diagram for the manufacture of the first generation famotidine granulate described in Example 1.

### DETAILED DESCRIPTION OF THE INVENTION

### Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “an active pharmaceutical agent” includes more than one active pharmaceutical agent, and the like.

“Pharmaceutically acceptable” means that which is useful in preparing a pharmaceutical composition that is generally safe, non-toxic and neither biologically nor otherwise undesirable and includes that which is acceptable for human pharmaceutical use.

“Therapeutically effective amount” means that amount which, when administered to an animal for treating a disease, is sufficient to effect such treatment for the disease. “Shelf stability”, for purposes of this invention, is measured by storing the dosage form in its primary packaging at 40° C., at a relative humidity of about. 75% for one, two and three months, or under 25° C. at a relative humidity of about 60% for three, six, nine, twelve, eighteen, twenty-four, thirty or thirty-six months. A stable formulation is one in which not more than about 0.5%, 1.0%, or 2.0% of the label claim of the 40 mg famotidine per dosage in the dosage form degrades.

When ranges are given by specifying the lower end of a range separately from the upper end of the range, it will be understood that the range can be defined by selectively combining any one of the lower end variables with any one of the upper end variables that is mathematically possible.

When used herein the term “about” or “ca.” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent in humans to the recited strength of a claimed product.

### Discussion

The invention provides a shelf-stable granulate mixtures of famotidine and excipients, including one or more organic or mineral acids, that can be mixed with water to form a suspension, resealed and stored for up to thirty days in ambient temperature and humidity. The granulate mixture is supplied dry in a sealed bottle, and mixed with water to provide a famotidine suspension that contains approximately 40 mg of famotidine for every 5 ml of water. In alternative embodiments, the formulation yields 35, 36, 37, 38, 39, 40, 41, 42, 43, 44, or 45 mg of famotidine for every 5 mL of water.

In a first embodiment, the invention provides a granulate for oral suspension comprising a first granulate and a second granulate, (a) said first granulate comprising famotidine in the absence of an organic or mineral acid; (b) said second granulate comprising one or more organic or mineral acids in the absence of famotidine; and (c) said first granulate and said second granulate being present at a ratio effective to produce a pH in water of from 6.5 to 7.5 when said granulate for oral suspension is mixed with water at a ratio of about 40 mg. of famotidine per 5 mL of water.

Another embodiment, which relates to the reconstitution of the granulate in water, provides a method of making an oral suspension comprising: (a) providing a granulate for oral suspension comprising a first granulate and a second granulate, (i) said first granulate comprising famotidine in the absence of an organic or mineral acid; (ii) said second granulate comprising one or more organic or mineral acids in the absence of famotidine; and (b) mixing said granulate for oral suspension with water at a ratio of about 40 mg of famotidine per 5 ml of water, in an amount effective to produce a pH in said water of from 6.5 to 7.5.

Yet another embodiment relates to the method for making the granulates, and in this embodiment, the invention provides method of making a granulate for oral suspension comprising: (a) granulating famotidine in the absence of an organic or mineral acid to produce a first granulate; (b) granulating one or more organic or mineral acids in the absence of famotidine to produce a second granulate; and (c) combining said first granulate and said second granulate to form a final granulate mixture, at a ratio effective to produce a pH in water of from 6.5 to 7.5 when said granulate for oral suspension is mixed with water at a ratio of about 40 mg of famotidine per 5 mL of water.

A second principal embodiment provides a granulate for oral suspension comprising a first granulate that comprises famotidine in combination with an organic or mineral acid, wherein said first granulate has the morphology of a dry granulate, said famotidine and organic or mineral acid being present at a ratio effective to produce a pH in water of from 6.5 to 7.5 when said granulate for oral suspension is mixed with water at a ratio of about 40 mg. of famotidine per 5 ml of water.

Another embodiment, which relates to the reconstitution of the dry granulate formulation in water, provides a method of making an oral suspension comprising: (a) providing a granulate for oral suspension comprising a first granulate that comprises famotidine in combination with an organic or mineral acid, wherein said first granulate has the morphology of a dry granulate; and (b) mixing said granulate for oral suspension with water at a ratio of about 40 mg of famotidine per 5 ml of water, in an amount effective to produce a pH in said water of from 6.5 to 7.5.

Yet another embodiment relates to the method for making the dray granulates, and in this embodiment, the invention provides a method of making a dry granulate for oral suspension comprising dry granulating famotidine and an organic or mineral acid at a ratio effective to produce a pH in water of from 6.5 to 7.5 when said granulate for oral suspension is mixed with water at a ratio of about 40 mg of famotidine per 5 mL of water.

The overall formulation for the preferred product is given below in Table A:

TABLE A

| Overall Composition | Sum | Optimized % w/w |
|---|---|---|
| Famotidine | 6.154 | 3.077 |
| Sugar (plus starch for | 188.957 | 94.4785 |
| Pilot) | | |
| Xanthan Gum | 2.360 | 1.18 |
| Sodium Benzoate | 0.770 | 0.385 |
| Sodium Methyl Paraben | 0.770 | 0.385 |
| Sodium Propylparaben | 0.154 | 0.077 |
| Mint Flavor | 0.120 | 0.06 |
| Cherry Flavor | 0.160 | 0.08 |
| Banana Flavor | 0.160 | 0.08 |
| Citric Acid | 0.400 | 0.20 |
| Total | 200.005 | 100% |

The product is preferably made in a three step process, defined as follows:

Step 1: famotidine is mixed with approximately ½ of the total quantity of xanthan gum and approximately ½ of the total quantity of sugar, and the mixture is granulated with a 0.5% xanthan gum solution

Step 2: approximately ½ of the total quantity of xanthan gum is mixed with approximately ½ of the total quantity of sugar, and the mixture is granulated with a solution of citric acid followed by a second granulation with a solution of the paraben preservatives (xanthan gum is present in the solution at 0.5%)

Step 3: Blend drug granules from step 1, preservative granules from step 2 and flavors

It is surprising that this three step process would result in a stable product because citric acid is not typically compatible with sodium salts of parabens.

The preferred acid for practicing the invention is an organic acid. An “organic acid” is an organic compound with acidic properties, or a salt thereof. The most preferred organic acids are the carboxylic acids whose acidity is associated with their carboxyl group —COOH. Sulfonic acids, containing the group OSO₃H, are relatively stronger acids, and less preferred. Sulfonic acids include methanesulfonic acid, ethanesulfonic acid, propanesulfonic acid and benzenesulfonic acid. The relative stability of the conjugate base of the acid determines its acidity. Other groups can also confer acidity, usually weakly: —OH, —SH, —N(R),(H)y (where x could be 0, 1, 2 and y=3−x), enol group, and the phenol group. For purposes of this invention, organic compounds containing only these groups are not generally referred to as organic acids. Exemplary organic acids are citric acid, malic acid, fumaric acid, propionic acid, stearic acid, tartaric acid, tannic acid, acetic acid, aconitic acid, dehydroacetic acid, folic acid, erythorbic acid, thiodipropionic acid, lactic acid, adipic acid, benzoic acid, ascorbic acid, sorbic acid, caprylic acid, linoleic acid, and any amino acid.

The sucrose can be any type of sugar including baker's grade (all pass through a 140 mesh screen), confectionary grade, and milled sucrose, and preferably is confectionary grade containing approximately 3-4 wt % starch.

The weight ratio of famotidine to citric acid in the final product is preferably from about 10:1 to about 20:1, and is more preferably from about 15:1 to about 16:1. The first granulate preferably comprises sugar at a sugar:famotidine weight ratio of from 5 to 25, more preferably from 10 to 20. The second granulate preferably comprises sugar at a sugar:organic acid weight ratio of from 40 to 500, more preferably from 100 to 300. The second granulate also preferably comprises one or more preservatives selected from sodium benzoate, sodium methyl paraben, sodium propyl paraben, and combinations thereof, at a preservative:organic acid weight ratio of from 1 to 20, or from 2 to 10.

The granulates also preferably comprise xanthan gum, and in a preferred embodiment the first granulate comprising xanthan gum at a xanthan gum: famotidine weight ratio of from 0.05 to 1.0, or from 0.1 to 0.5. The second granulate preferably comprises xanthan gum at a xanthan gum:organic acid weight ratio of from 0.5 to 10, or from 1 to 5.

### EXAMPLES

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at room temperature, and pressure is at or near atmospheric.

### Example 1—First Generation Granulate Formulation

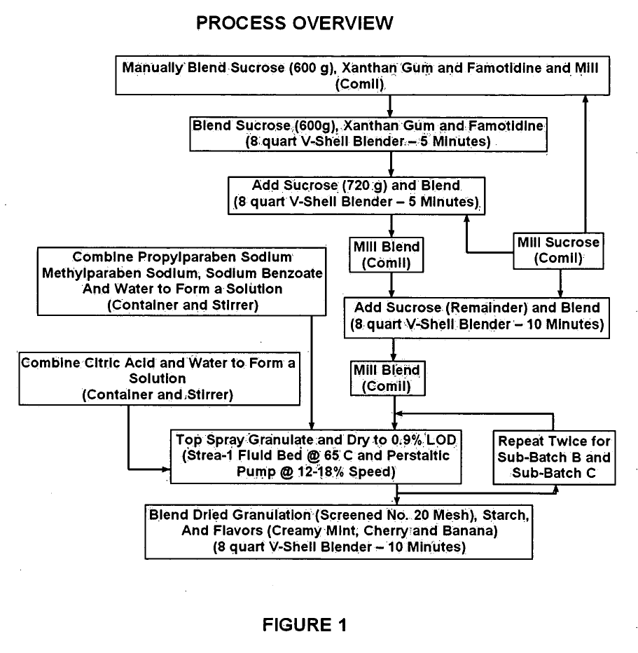

Table 1 reports the ingredients of a famotidine for oral suspension granulate initially made by the inventors. FIG. 1 contains a flow chart for the manufacturing process. The granulate produced by this process was robust in terms of handling, manufacturability and its ability to form an oral suspension. However, the product was not sufficiently shelf-stable when stored for prolonged periods.

TABLE 1

| | | | % w/w | Amount (g) | |
|---|---|---|---|---|---|
| Component | g/Bottle | | of Blend | for 3,000 g | |
| Granulation Medium 1 | | | | | |
| Methylparaben Sodium, NF** | 0.055 | | 0.385 | 11.538 | |
| (27261) | | | | | |
| Propylparaben Sodium, NF** | 0.011 | | 0.077 | 2.308 | |
| (27262) | | | | | |
| Sodium Benzoate, NF | 0.055 | | 0.385 | 11.538 | |
| (27260) | | | | | |
| Citric Acid, Anhydrous, USP | 0.029 | | 0.200 | 6.000 | |
| (27255) | | | | | |
| Purified Water, USP* | | | | 180.00 | |
| (23004) | | | | | |
| Total Granulation Medium | | | | 211.384 | |
| Total Granulation Medium | | | | 31.384 | |
| Solids | | | | | |
| Granulation Powder Blend | | | | | |
| Famotidine, USP | 0.440 | | 3.077 | 92.31 | |
| (Gideon Richter, 27232) | | | | | |
| Xanthan Gum, NF | 0.169 | | 1.180 | 35.40 | |
| (Xantural 75, 27264) | | | | | |
| Sucrose, NF | 13.081 | | 91.476 | 2744.31 | |
| (27289) | | | | | |
| Total Granulation Powder Blend | | | | 2872.02 | |
| Final Blend*** | | | | | |
| Starch, Powder, NF | 0.429 | | 3.000 | 90.000 | |
| (UniPure FL, 27256) | | | | | |
| Flavored Powder, Creamy Mint | 0.009 | | 0.060 | 1.800 | |
| (27259) | | | | | |
| Flavored Powder, Cherry | 0.011 | | 0.080 | 2.400 | |
| (27258) | | | | | |
| Flavored Powder, Banana | 0.011 | | 0.080 | 2.400 | |
| (27257) | | | | | |
| Total Final Blend | | | | 96.600 | |
| Total | 14.300 | g | 100.000 | 3000.00 | g |

*To be removed during drying.
**Actual amount dispensed to be determined by loss on drying.
***The Amount/Batch of the excipients dispensed for the Final Blend will be adjusted for the true recovery of the Granulation after drying and sieving.

### Example 2—Final Granulate Formulation

Tables 2 and 3 report the ingredients of two separate granulates used in the optimal combination of granulates used in the final commercial formulation. Table 4 reports the amounts of ingredients contained in the final formulation.

TABLE 2
Active Ingredient Granulate Ingredients

| No. | Compound | Wt % | Wt (g) | Supplier |
|---|---|---|---|---|
| 1 | Confectionary Sugar - | 5.000 | 35.000 | Imperial |
| | 6X | | | |
| 2 | Famotidine | 6.154 | 43.078 | Gideon Richter |
| 3 | Xanthan Gum | 1.150 | 8.050 | CP Kelco |
| 4 | Confectionary Sugar - | 7.500 | 52.500 | Imperial |
| | 6X | | | |
| 5 | Confectionary Sugar - | 15.000 | 105.000 | Imperial |
| | 6X | | | |
| 6 | Confectionary Sugar - | 30.000 | 210.000 | Imperial |
| | 6X | | | |
| 7 | Confectionary Sugar - | 35.167 | 246.169 | Imperial |
| | 6X | | | |
| 8 | Xanthan Gum | 0.030 | 0.210 | CP Kelco |
| 9 | DI Water | 6.000 | 42.000 | NA |
| 10 | DI Water | 0.500 | 3.500 | NA |
| | Total | 100.00 | 700.01 | |

*6X Confectioner's Sugar (Lot C226G) includes 3.100% corn starch.
Steps for Making API Granulate
1.) Combine compounds (1), (2), and (3) to a robot coupe and mix for 2 minutes at 1500 rpm.
2.) Add compound (4) and mix for 1 minute at 1500 rpm.
3.) Add compound (5) and mix for 1 minute at 1500 rpm.
4.) Add compound (6) and mix for 1 minute at 1500 rpm.
5.) Add compound (7) and mix for 1 minute at 1500 rpm.
6.) Add compound (9) to a beaker and while stirring with an overhead stirrer, slowly add the mixture from compound (8) until dissolved.
7.) Granulate the mixture from step 5 with the solution prepared in step 6 at 1500 rpm. Add solution over 30 seconds and continue mixing for 1 minute.
8.) Disperse granulation in tray and break up large agglomerates before placing back in robot coupe.
9.) Rinse beaker with compound (10) and granulate with this solution. Add solution over 30 seconds and continue granulating for 1 minute.
10.) Sieve granulation through 20 mesh screen while wet.
11.) Dry the granulation in oven at 55° C. until an LOD (@105° C.) of less than 0.90% is obtained.
12.) Pass the granulation through a 20 mesh stainless steel screen.
13.) Place granulation in an amber bag until mixed with G2 or dispensed into bottles.

TABLE 3
Citric Acid Granulate Ingredients

| No. | Compound | Wt % | Wt (g) | Supplier |
|---|---|---|---|---|
| *1 | Confectionary Sugar - 6X | 5.000 | 35.000 | Imperial |
| 2 | Famotidine | 0.000 | 0.000 | Gideon Richter |
| 3 | Xanthan Gum | 1.152 | 8.064 | CP Kelco |
| 4 | Confectionary Sugar - 6X | 7.500 | 52.500 | Imperial |
| 5 | Confectionary Sugar - 6X | 15.000 | 105.000 | Imperial |
| 6 | Confectionary Sugar - 6X | 30.000 | 210.000 | Imperial |
| 7 | Confectionary Sugar - 6X | 38.790 | 271.530 | Imperial |
| **8 | Sodium Benzoate | 0.770 | 5.390 | Spectrum |
| **9 | Sodium Methyl Paraben | 0.770 | 5.390 | Clariant |
| **10 | Sodium Propylparaben | 0.154 | 1.078 | Clariant |
| 11 | DI Water | 3.000 | 21.000 | NA |
| 12 | Xanthan Gum | 0.014 | 0.098 | CP Kelco |
| 13 | DI Water | 0.500 | 3.500 | NA |
| 14 | Citric Acid | 0.400 | 2.800 | Spectrum |
| 15 | Xanthan Gum | 0.014 | 0.098 | CP Kelco |
| 16 | DI Water | 2.500 | 17.500 | NA |
| 17 | DI Water | 0.500 | 3.500 | NA |
| 18 | Mint Flavor | 0.120 | 0.840 | Cargill |
| 19 | Cherry Flavor | 0.160 | 1.120 | Cargill |
| 20 | Banana Flavor | 0.160 | 1.120 | Cargill |
| | Total | 100.00 | 699.93 | |

*6 X Confectioner's Sugar (Lot C226G) includes 3.100% corn starch.
Steps for Making Citric Acid Granules
1.) Combine compounds (1), (2), and (3) to a robot coupe and mix for 2 minutes at 1500 rpm.
2.) Add compound (4) and mix for 1 minute at 1500 rpm.
3.) Add compound (5) and mix for 1 minute at 1500 rpm.
4.) Add compound (6) and mix for 1 minute at 1500 rpm.
5.) Add compound (7) and mix for 1 minute at 1500 rpm.
6.) Add compounds (8), (9), (10), and (11) to a beaker while stirring with an overhead stirrer. Slowly add compound (12) to the solution.
7.) Granulate the mixture from step 5 with the solution prepared in step 6 at 1500 rpm. Add solution over 1 minute and continue mixing for 1 minute.
8.) Rinse beaker with compound (13) and granulate with this solution. Add solution over 30 seconds and continue granulating for 1 minute.
9.) Add compounds (14), (15), and (16) to a beaker and stir until homogeneous.
10.) Granulate the mixture from step 8 with the solution from step 9. Add solution over 1 minute and continue mixing for 1 minute.
11.) Rinse beaker with compound (17) and granulate with this solution. Add solution over 30 seconds and continue mixing for 1 minute.
12.) Pass the granulation through a 20 mesh stainless steel screen.
13.) Dry the granulation in oven at 55 C. until an LOD (@105° C.) of less than 0.90% is obtained.
14.) Pass the granulation through a 20 mesh stainless steel screen.
15.) To the granulate from step (14). Add compounds (18), (19), and (20) and mix in V-shell blender for 300 revolutions.
16.) Place granulation from step (15) in an amber bag until mixed with G1 prior to dispensing into bottles.

TABLE 4
Final Formulation

| Overall Composition | Sum | Optimized % w/w |
|---|---|---|
| Famotidine | 6.154 | 3.077 |
| Sugar (plus starch for | 188.957 | 94.4785 |
| Pilot) | | |
| Xanthan Gum | 2.360 | 1.18 |
| Sodium Benzoate | 0.770 | 0.385 |
| Sodium Methyl Paraben | 0.770 | 0.385 |
| Sodium Propylparaben | 0.154 | 0.077 |
| Mint Flavor | 0.120 | 0.06 |
| Cherry Flavor | 0.160 | 0.08 |
| Banana Flavor | 0.160 | 0.08 |
| Citric Acid (amount added | 0.400 | 0.200 |
| prior to granulation) | | |
| Total | 200.005 | 100 |

### Example 3—Stability Study

Table 5 displays the results of stability testing for the formulations prepared according to Example 2. The stability data impurities test was done according to the method as found in the monograph “Famotidine Suspension” as found in the United States Pharmacopeia 30. Surprisingly compared to the first generation granulate formulation, for formulation of example 2 the impurities are extremely low.

TABLE 5
Stability-Testing Impurity Results for Formulation of Example 2

| | | | Blend of Two Granules | |
|---|---|---|---|---|
| | | Preservatives | (Famotidine Granules | Powder blend |
| | Famotidine | Granules Blend | and Preservative | of famotidine |
| Composition | Granules | w/ Flavors | Granules) | and sugar |
| Initial | None detected | None detected | None detected | None detected |
| 1 month at | 0.06%, 0.14% | None detected | 0.08%, 0.13% | < LQ, 0.14% |
| 40° C./75% RH | | | | |
| 2 months at | 0.06%, 0.12% | None detected | 0.17% | 0.05%, 0.13% |
| 40° C./75% RH | | | | |
| 3 months at | 0.06%, 0.16% | None detected | 0.22%, 0.16% | < LOQ, 0.17% |
| 40° C./75% RH | | | | |

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A granulate for oral suspension comprising a first granulate and a second granulate,
a) said first granulate comprising famotidine in the absence of an organic or mineral acid;
b) said second granulate comprising one or more organic or mineral acids, in the absence of famotidine;
c) said first granulate and said second granulate being present at a ratio effective to produce a pH in water of from 6.5 to 7.5 when said granulate for oral suspension is mixed with water at a ratio of about 40 mg. of famotidine per 5 ml of water.

2) The granulate for oral suspension of claim 1 comprising famotidine and citric acid at a weight ratio of from 10:1 to 20:1.

3) The granulate for oral suspension of claim 1 comprising famotidine and citric acid at a weight ratio of from 15:1 to 16:1.

4) The granulate for oral suspension of claim 1 wherein said first granulate and said second granulate are wet granulated with an aqueous solution of xanthan gum.

5) The granulate for oral suspension of claim 1,
a) said first granulate comprising sugar at a sugar:famotidine weight ratio of from 5 to 25;
b) said second granulate comprising sugar at a sugar:organic acid weight ratio of from 100 to 500;
c) said second granulate comprising one or more preservatives selected from sodium benzoate, sodium methyl paraben, sodium propyl paraben, and combinations thereof, at a preservative:organic acid weight ratio of from 1 to 20.

6) The granulate of claim 1,
a) said first granulate comprising xanthan gum at a xanthan gum: famotidine weight ratio of from 0.05 to 1.0;
b) said second granulate comprising xanthan gum at a xanthan gum:organic acid weight ratio of from 0.5 to 10.

7) A method of making an oral suspension comprising:
a) providing a granulate for oral suspension comprising a first granulate and a second granulate,
i) said first granulate comprising famotidine in the absence of an organic or mineral acid;
ii) said second granulate comprising one or more organic or mineral acids in the absence of famotidine;
b) mixing said granulate for oral suspension with water at a ratio of about 40 mg of famotidine per 5 ml of water, in an amount effective to produce a pH in said water of from 6.5 to 7.5.

8) The method of claim 7 wherein said granulate for oral suspension comprises famotidine and citric acid at a weight ratio of from 10:1 to 20:1.

9) The method of claim 7 wherein said granulate for oral suspension comprises famotidine and citric acid at a weight ratio of from 15:1 to 16:1.

10) The method of claim 7 wherein said first granulate and said second granulate are wet granulated with an aqueous solution of xanthan gum.

11) The method of claim 7 wherein,
a) said first granulate comprising sugar at a sugar:famotidine weight ratio of from 5 to 25;
b) said second granulate comprising sugar at a sugar:organic acid weight ratio of from 100 to 500;
c) said second granulate comprising one or more preservatives selected from sodium benzoate, sodium methyl paraben, sodium propyl paraben, and combinations thereof, at a preservative:organic acid weight ratio of from 1 to 20.

12) The method of claim 7 wherein,
a) said first granulate comprising xanthan gum at a xanthan gum: famotidine weight ratio of from 0.05 to 1.0;
b) said second granulate comprising xanthan gum at a xanthan gum:organic acid weight ratio of from 0.5 to 10.

13) A method of making a granulate for oral suspension comprising:
a) granulating famotidine in the absence of an organic or mineral acid to produce a first granulate;
b) granulating one or more organic or mineral acids in the absence of famotidine to produce a second granulate; and
c) combining said first granulate and said second granulate to form a final granulate mixture, at a ratio effective to produce a pH in water of from 6.5 to 7.5 when said granulate for oral suspension is mixed with water at a ratio of about 40 mg. of famotidine per 5 ml of water.

14) The method of claim 13, comprising:
a) wet granulating a dry mixture of said famotidine, xanthan gum and sugar in a first aqueous solution or dispersion of xanthan gum to produce said first granulate, and
b) wet granulating a dry mixture of xanthan gum and sugar in a second aqueous solution or dispersion of xanthan gum and citric acid to produce an intermediate granulate;
c) wet granulating said intermediate granulate in a third aqueous solution or dispersion of xanthan gum and one or more sodium parabens to produce said second granulate.

15) The method of claim 14 comprising wet granulating a dry mixture of xanthan gum and sugar in a second aqueous solution or dispersion of xanthan gum, sodium benzoate, sodium methyl paraben and sodium propyl paraben.

16) The method of claim 14 further comprising combining said final granulate mixture with one or more flavorings selected from mint flavor, cherry flavor and banana flavor.

17) A granulate for oral suspension comprising a first granulate that comprises famotidine in combination with an organic or mineral acid, wherein said first granulate has the morphology of a dry granulate, said famotidine and organic or mineral acid being present at a ratio effective to produce a pH in water of from 6.5 to 7.5 when said granulate for oral suspension is mixed with water at a ratio of about 40 mg. of famotidine per 5 ml of water.

18) A method of making an oral suspension comprising: (a) providing a granulate for oral suspension comprising a first granulate that comprises famotidine in combination with an organic or mineral acid, wherein said first granulate has the morphology of a dry granulate; and (b) mixing said granulate for oral suspension with water at a ratio of about 40 mg of famotidine per 5 ml of water, in an amount effective to produce a pH in said water of from 6.5 to 7.5.

19) A method of making a dry granulate for oral suspension comprising dry granulating famotidine and an organic or mineral acid at a ratio effective to produce a pH in water of from 6.5 to 7.5 when said granulate for oral suspension is mixed with water at a ratio of about 40 mg of famotidine per 5 mL of water.
